(19) United States
(12) Patent Application Publication
(10) Pub. No.: US 2012/0071425 A1
(43) Pub. Date: Mar. 22, 2012

(54) USES OF MANNOSE-6-PHOSPHATE

(75) Inventors: Mark William James Ferguson, Manchester (GB); Sharon O'Kane, Manchester (GB); Nicholas Occleston, Manchester (GB); Kerry Nield, Manchester (GB); Tracey Mason, Manchester (GB); James Bush, Manchester (GB); Julian Hanak, Manchester (GB)

(73) Assignee: RENOVO LIMITED, Manchester (GB)

(21) Appl. No.: 13/322,660

(22) PCT Filed: Jun. 10, 2010
(86) PCT No.: PCT/GB2010/001137

§ 371 Date: Nov. 28, 2011

(30) Foreign Application Priority Data
Jun. 11, 2009 (GB) 0910078.5

(51) Int. Cl. A61K 8/60; A61Q 19/00; A61P 17/00; C07H 11/04; A61K 31/7024

(52) U.S. Cl. 514/23; 536/117

(57) ABSTRACT

There is provided the use of mannose-6-phosphate, or a salt, precursor or analogue thereof, for providing and/or maintaining a consistent skin colour, particularly to reducing redness of skin. There is also provided the use of mannose-6-phosphate, or a salt, precursor or analogue thereof, as a skin improvement agent for providing a cosmetic effect and mannose-6-phosphate, or a salt, precursor or analogue thereof, for use in treating normal or damaged skin, wherein damaged skin is skin that has been subject to epidermal and/or dermal damage.

The present invention relates to the use of mannose-6-phosphate, or a salt, precursor or analogue thereof, for providing and/or maintaining a consistent skin colour, particularly for reducing redness of skin. The present invention further relates to the use of mannose-6-phosphate, or a salt, precursor or analogue thereof, as a skin improvement agent for providing a cosmetic effect, and to mannose-6-phosphate, or a salt, precursor or analogue thereof, for use in treating normal or damaged skin, wherein damaged skin is skin that has been subject to epidermal and/or dermal damage.

Skin is composed of three primary layers, the epidermis, the dermis and the hypodermis. The dermis is a layer between the epidermis and subcutaneous tissues and is composed of two layers, the papillary and reticular dermis. The outermost layer of the skin is the epidermis, which forms a waterproof, protective wrap over the surface of the body. The epidermis is composed of four to five layers, depending on the region of the skin being considered. These layers, in descending order, are the stratum corneum, stratum lucidum, stratum granulosum, stratum spinosum and stratum basale. The stratum corneum, as the outermost layer of the skin, is composed of cells that have been generated in and migrated from the stratum basale, during which process the cells produce and accumulate keratin. Upon reaching the stratum corneum, the cells contain virtually no cytoplasm and die and are sloughed off (or shed) from the skin. This process is ongoing and in healthy, young skin the continual sloughing of cells from the surface maintains the skin and leaves it looking smooth and fresh. This is of course desirable, as the skin then maintains a youthful, smooth and even appearance.

However, if the sloughing process is interrupted or does not perform efficiently, dead cells can accumulate in the stratum corneum and result in an undesirable appearance and feel of the skin. For example, the accumulation of dead cells in the stratum corneum results in a dull, patchy, irregular feel and appearance of the skin. Interruptions to and inefficiency in sloughing typically occurs as skin ages and biological processes begin to slow down and become less efficient. The problems associated with such ageing processes are of course more readily observable in the area of the face, neck and/or hand, especially the face and/or neck, more especially the face, and may cause distress to persons as they age.

In order to overcome the problems caused by interrupted or inefficient sloughing of skin cells, cosmetic products and methods have been developed to accelerate and/or supplement the sloughing process. These known cosmetic products and methods include exfoliants. An exfoliant is an agent or substance used for the process of exfoliation, i.e. the removal of dead skin cells from the stratum corneum, either physically by means of abrasive ingredients or implements (such as scrubs or cleansing pads) or chemically, for example using enzymes or acids. The removal of dead skin cells from the stratum corneum may also occur by means of tape stripping, shaving or waxing.

Other known cosmetic products and methods for accelerating and/or supplementing the sloughing process include various skin resurfacing techniques. Skin resurfacing means a procedure whereby the epidermis or both the epidermis and the dermis of the skin is injured by a skin peel procedure, for example by use of a chemical skin peel agent, a laser (i.e. laser peel) or dermabrasion, with the purpose of removing superficial lesions and/or to improve skin texture.

In contrast to exfoliants, skin resurfacing procedures remove both dead skin cells from the stratum corneum and live skin cells from below the stratum corneum (i.e. from the stratum lucidum and below). Thus, skin resurfacing procedures act to remove outer skin layers so as to leave a new layer of fresh skin visible at the surface of the skin, which fresh skin provides a fresh, youthful appearance of the skin.

The injury and removal of the epidermis and dermis is provided by an active chemical ingredient in a chemical skin peel agent or by an appropriate laser in a laser treatment. The penetration level of laser or chemical skin peel agent and the nature of destruction determines the depth of the resurfacing. Skin resurfacing procedures are classified as superficial, medium and deep. The selection of a particular skin resurfacing procedure, for example specific chemical skin peel agent or laser type, and the depth of the procedure is driven by the cosmetic outcome desired, the subject's physiology (for example skin type) and constraints, such as the recovery period (“downtime”) the subject is willing to undergo and cost. For example, medium procedures may be repeated every three to six months for optimal results and (unlike deep treatments) may be performed safely on people with olive and light brown skin. They may also be used in some people with dark brown skin, although the risk of discoloration is higher.

Another known skin resurfacing procedure is dermabrasion (including microdermabrasion), whereby layers of the epidermis and often dermis are removed by mechanical means. Dermabrasion uses a power-driven hand held device with a rotating metal wheel, diamond fraise or wire brush to remove the skin. Microdermabrasion uses a device comprising a compressor and pump that mixes gentle abrasion with suction to remove an outer skin layer, typically the epidermal layer.

The popularity of the skin resurfacing procedures discussed above is growing in response to patient demand for procedures that improve skin tone and appearance, but are less invasive than traditional cosmetic surgery, such as facelifts. Thus, there is a large demand for such treatments that can improve the appearance of skin in a convenient manner. Typically, skin resurfacing procedures are currently conducted on the skin of the face, neck and hand, although they may be used on any area of the skin where an improvement is desired.

Numerous chemical skin peel agents are known and are comprised of active chemical ingredients or active biological/enzymatic ingredients that peel away or remove the outer layers of the skin. Typically, chemical skin peel agents are simply applied to the skin (especially the face) in a non-surgical cosmetic procedure. Application may be by means of any suitable applicator, such as a sponge, cotton pad, cotton swab or brush, to the area(s) to be treated, avoiding sensitive areas such as the eyes, brows and lips.

Chemical skin peel agents vary according to their specific ingredients and strength. Depth of peeling action may also depend on factors such as how long the agent remains on the skin and whether it is lightly applied or more heavily or vigorously applied.

Superficial chemical skin peel agents typically comprise active chemical ingredients such as alpha hydroxy acids (AHAs), for example glycolic, lactic or fruit acid, or beta hydroxy acids (BHAs). Medium chemical skin peel agents typically comprise trichloroacetic acid (TCA) and deep chemical skin peel agents typically comprise phenol as the active chemical ingredient. The active chemical ingredient effectively burns the skin and kills skin cells to the desired depth, causing the cells to then peel (or shed) from the skin. Some active chemical ingredients are believed also to stimulate the production of collagen and the growth of better quality skin.

Numerous laser skin resurfacing treatments are also known. Laser skin resurfacing is a process in which the upper layers of the skin are vaporised by applying a controlled laser beam. The two most commonly used lasers for wrinkle removal are carbon dioxide and erbium:YAG lasers. Medium laser treatments typically are conducted using fractionated lasers (for example, fraxel, pearl or carbon dioxide lasers) and deep laser treatments typically are conducted using ablative lasers (for example carbon dioxide or erbium:YAG lasers). Another important technical aspect is the number of passes made when treating skin with a laser. Multiple passes, particularly with a carbon dioxide laser, generally produce greater thermal injury and lead to greater side effects and longer recovery times. Laser skin resurfacing procedures typically are used on either the entire face, or, more commonly, on the areas around the eyes and mouth and may provide a greater precision compared, for example, to chemical skin peel agents.

As discussed above, skin resurfacing procedures are intended to provide a fresh, youthful appearance to the skin. The procedures are believed to help to improve fine lines in the skin (for example by improving laughter lines, crow's feet, brow lines and crinkly skin), acne damage (for example by reducing or removing dark marks, red spots and rough skin), sun damage (for example by reversing and removing photo-aged and sun-damaged skin), age spots (for example by removing or lightening of dark spots and blotches), uneven skin tone (for example by lightening and brightening to provide an even coloured skin tone), hyper-pigmentation (for example by reducing and improving the appearance of darkened skin patches and spots) and other complexion irregularities.

Whilst skin resurfacing procedures are used to improve the appearance of the skin, there are some unpleasant side effects to their use. Upon conducting a skin resurfacing procedure, a stinging or burning sensation can result and areas of redness typically appear on the skin on which the procedure has been conducted. Such redness may particularly be a problem associated with medium and deep penetrating skin resurfacing procedures, where the skin redness can be severe and long lasting. For example, significant redness may be caused by medium and especially deep chemical skin peel agents and lasers. Other side effects that can occur upon conducting a skin resurfacing procedure include hypo-pigmentation, hyper-pigmentation and infection of the skin.

Following a deep chemical skin peel procedure (for example with a chemical skin peel agent comprising phenol), the face typically is swollen, red, oozing, and crusted. Over several days, the face will become stiff, dark, and begin to flake. Within seven to ten days, the peeling and flaking will be complete and the face will be healthy, smooth, bright red skin. This sunburned appearance takes two to three months to improve and the skin can then appear extremely pale, such that make-up is required in order to maintain a normal, presentable appearance.

Following a medium chemical skin peel procedure (for example with a chemical skin peel agent comprising trichloroacetic acid), the skin will initially appear red. Within a few days, the superficial layers of skin will turn dark, become stiff, and resemble leather. They will then crack, flake, and peel. Flaking is usually complete in four to seven days and there will be no open wounds and no scabs. Once the old skin has sloughed, the new skin will be bright and flushed. Once the skin has finished peeling, the subject may begin wearing makeup although redness will take several weeks to fade.

Following a laser skin peel, the skin reddens and becomes dry. Subjects with darker skin are more likely to develop uneven pigmentation.

Skin care after a deep skin resurfacing procedure is onerous for the subjects and relatively time intensive for the after care/cosmetic procedure clinic. For example, immediately following a deep skin resurfacing procedure, it typically is necessary to apply ice packs to the treated skin to reduce swelling, to apply a bio-occlusive petroleum based product four to ten times daily to avoid skin drying and ensure successful healing of the skin, and to wash the skin at least four times daily with a very mild cleanser (for example Cetaphil) or dilute acetic acid solution to remove exudate, assist skin sloughing and discourage bacterial/fungal growth. In the days following a deep skin resurfacing procedure, it typically is necessary to attend a clinic for the skin to be checked for signs of infection or other complications and in the weeks following the procedure, a moisturising cream (for example comprising skin nutrients) needs to be applied to the skin, and make-up is required to cover redness and enable the subject to resume normal activities.

It would of course be desirable to provide alternative skin treatments for treating skin, especially damaged skin, and for providing cosmetic improvements to skin, for example by improving the visual appearance and/or feel of the skin. For example, it is desirable to provide such treatments whereby the treatments do not suffer from the unpleasant side effects associated with skin resurfacing procedures discussed above. Additionally, it is desirable to provide alternative skin treatments that may be used either alone or in combination with presently known treatments, such as skin resurfacing procedures. Such alternative, and especially improved, skin treatments are particularly desired for use on the face, neck and hand, more particularly on the face and neck and even more particularly on the face, although they may of course be useful on the skin on any area of the body where treatment and improvement is desired.

According to the present invention, there is provided mannose-6-phosphate, or a pharmaceutically or cosmetically acceptable salt, precursor, or analogue thereof, for use in reducing redness of skin.

The skin may have been subject to damage, particularly damage to the epidermis and optionally the dermis. The damage may have resulted from a skin resurfacing procedure.

The mannose-6-phosphate, pharmaceutically or cosmetically acceptable salt, precursor, or analogue thereof may be formulated as a cosmetic composition, for example, a cosmetic composition for application to the skin by topical administration.

The present invention further provides mannose-6-phosphate, or a pharmaceutically or cosmetically acceptable salt, precursor, or analogue thereof, for use in providing and/or maintaining a consistent skin colour.

Another aspect of the present invention provides mannose-6-phosphate, or a pharmaceutically or cosmetically acceptable salt, precursor, or analogue thereof, for use as a skin improvement agent for providing a cosmetic effect. The skin improvement agent may improve macroscopic properties of the skin and/or improve the visual appearance and/or feel (especially visual appearance) of the skin. The skin improvement agent may improve skin that has been subject to damage, particularly damage to the epidermis and optionally the dermis, damage which may have resulted from a skin resurfacing procedure. Moreover, the skin improvement agent may provide and/or maintain a consistent skin colour, and/or reduces redness of the skin. The mannose-6-phosphate, salt, precursor, or analogue may be formulated as a cosmetic composition, in particular a cosmetic composition for application to the skin by topical administration.

Mannose-6-phosphate (also referred to as “M6P”) is a well-known low molecular weight monosaccharide and is known for use in the medical treatment of fibrotic disorders that give rise to deleterious or pathological structures within the skin. For example, WO-93/18777 discloses the use of mannose-6-phosphate in the medical treatment of fibrotic disorders, such as skin wounds, and for accelerating wound healing and preventing or mitigating scar formation. WO-98/06406 discloses improved compositions comprising mannose-6-phosphate for use in the medical treatment of similar fibrotic disorders.

Both WO-93/18777 and WO-98/06406 are focused upon the application of mannose-6-phosphate to the treatment of fibrotic disorders that give rise to deleterious or pathological structures within the skin, such as scars, or other sites of fibrosis. The skilled person will appreciate that such conditions are quite distinct from the types of conditions to which the present invention is directed, which are related to the cosmetic properties of the skin, in particular reducing redness of the skin and/or improving the appearance of skin. Various aspects of the present invention thus relate to cosmetic applications of mannose-6-phosphate, which are clearly quite different to the types of fibrotic disorders targeted in WO-93/18777 and WO-98/06406. Moreover, aspects of the present invention are directed to the therapeutic use of mannose-6-phosphate in the treatment of skin redness to reduce the appearance of the redness.

WO-03/013448 discloses the use of phosphosugars, including mannose-6-phosphate, as exfoliants and for increasing levels of glycosaminoglycans in skin cells. Whilst there is some discussion in WO-03/013448 in relation to possible changes to skin properties that may result from exfoliating or increasing glycosaminoglycan levels, there is no evidence of any such changes provided. Furthermore, WO-03/013448 neither mentions nor suggests the use of mannose-6-phosphate to treat or reduce the redness of skin.

According to the present invention, there is provided the use of mannose-6-phosphate, or a salt, precursor, or analogue thereof, for providing and/or maintaining a consistent skin colour.

According to the present invention, there is also provided a method for providing and/or maintaining a consistent colour of the skin of a subject, wherein the method comprises the step of administering an effective amount of mannose-6-phosphate, or a salt (especially a cosmetically acceptable salt), precursor, or analogue thereof, to the skin of the subject.

According to the present invention, there is provided the use of mannose-6-phosphate, or a salt (especially a cosmetically acceptable salt), precursor, or analogue thereof, for reducing redness of skin.

According to the present invention, there is also provided a method for reducing redness of the skin of a subject, wherein the method comprises the step of administering an effective amount of mannose-6-phosphate, or a salt (especially a cosmetically acceptable salt), precursor, or analogue thereof, to the skin of the subject.

According to the present invention, there is provided the use of mannose-6-phosphate, or a cosmetically acceptable salt, precursor, or analogue thereof, as a skin improvement agent for providing a cosmetic effect.

According to the present invention, there is also provided a method for providing a cosmetic effect by improving the skin of a subject, wherein the method comprises the step of administering an effective amount of mannose-6-phosphate, or a cosmetically acceptable salt, precursor, or analogue thereof, to the skin of the subject.

According to the present invention, there is also provided the use of mannose-6-phosphate, or a cosmetically acceptable salt, precursor, or analogue thereof, as a cosmetic product (for example for a cosmetic purpose).

According to the present invention, there is also provided a method for providing a cosmetic effect to the skin of a subject, wherein the method comprises the step of administering an effective amount of mannose-6-phosphate, or a salt (especially a cosmetically acceptable salt), precursor, or analogue thereof, to the skin of the subject.

According to the present invention, there is also provided the use of mannose-6-phosphate, or a cosmetically acceptable salt, precursor, or analogue thereof, as an active agent in a cosmetic composition.

According to the present invention, there is also provided the use of mannose-6-phosphate, or a cosmetically acceptable salt, precursor, or analogue thereof, as a skin improvement agent in a cosmetic composition.

According to the present invention, there is also provided a method for providing a cosmetic effect to the skin of a subject, wherein the method comprises the step of administering an effective amount of a cosmetic composition, wherein the cosmetic composition comprises mannose-6-phosphate, or a cosmetically acceptable salt, precursor, or analogue thereof, to the skin of the subject.

According to the present invention, there is also provided a cosmetic method for improving the skin of a subject, wherein the method comprises the step of administering an effective amount of mannose-6-phosphate, or a cosmetically acceptable salt, precursor, or analogue thereof, to the skin of the subject.

According to the present invention, there is further provided a cosmetic method for improving the skin of a subject, wherein the method comprises the step of administering an effective amount of mannose-6-phosphate, or a cosmetically acceptable salt, precursor, or analogue thereof, to the skin of the subject so as to provide a cosmetic effect to the skin.

The present invention provides a skin treatment for providing cosmetic improvements to skin, for example by improving the visual appearance and/or feel of the skin, which does not suffer from the unpleasant side effects associated with skin resurfacing procedures discussed above and therefore is convenient to use on a regular basis.

By a “cosmetic effect” we mean an effect that is designed to beautify and improve the overall appearance of a subject, particularly that is designed to beautify and improve the skin and/or complexion of the subject, and which is a non-medical effect. In other words, a cosmetic effect provides a cosmetically beneficial improvement to the skin and is not beneficial to the physical health of the subject. The cosmetic effect may be provided to any area of the body of the subject, which area requires cosmetic treatment or improvement. In particular, the cosmetic effect may be provided to the face, neck and/or hand, especially to the face and/or neck, more especially to the face, of the subject.

By a “cosmetic method” we mean a method that provides a cosmetic effect as discussed herein.

By a “skin improvement agent” we mean an agent that is effective to provide a beneficial effect to the skin, which beneficial effect is intended to beautify and improve the skin and/or complexion, when it is administered to a subject. By “improving the skin” we mean providing a beneficial effect to the skin as discussed herein, wherein the beneficial effect is a non-medical effect. References to improving the skin include improving problem aspects of the skin, as well as guarding against future problems associated with the skin of a subject. The improvement may be provided to any region of the skin where an improvement is desired, although in particular, the improvement may be provided to the skin of the face, neck and/or hand, especially of the face and/or neck, more especially of the face, of the subject. In the case where the skin improvement agent of the present invention is administered to an area of skin of a subject that has been subject to a prior procedure (for example a skin resurfacing procedure), the improvement may be considered to mean an improvement compared to an area of the skin of the same subject that has not been subject to the prior procedure (preferably to an area of the skin in the same region of the body).

In particular, the skin improvement agent improves macroscopic properties of the skin. As the skilled person would appreciate, by macroscopic properties we mean properties that are measurable and observable to the naked eye. For example, the skin improvement agent preferably improves the visual appearance of the skin.

Typically, the mannose-6-phosphate as a skin improvement agent improves one or more of the following properties of the skin:
(i) evens out and improves skin tone
(ii) provides lift (preferably instant lift)
(iii) provides hydration
(iii) gives skin a smooth, youthful appearance
(iv) re-plumps and replenishes skin (for example to provide immediate lift)
(v) re-textures skin
(vi) recovers and brightens skin
(vii) protects skin
(viii) reduces the appearance of dark spots
(ix) calms and reduces redness
(x) repairs an ageing appearance, for example by improving or effectively removing wrinkles (i.e. fine lines and/or deeper wrinkles) and/or age spots
(xi) moisturises skin
(xii) guards against the premature visible aging effects of sun
(xiii) improves clarity
(xiv) improves radiance
(xv) improves colour
(xvi) improves freshness
(xvii) improves blotches
(xviii) improves and/or removes dark areas, such as dark circles under the eyes
(xix) softening the skin
(xx) improves acne damage
(xxi) soothes and calms the skin
(xxii) eases the effects of sunburn
(xxiii) soothes nappy rash in babies
(xxiv) calms skin subject to shaving irritation
(xxv) treats the undesirable cosmetic side-effects following a skin peel
(xxvi) soothes skin after exfoliation

References to improving the visual appearance of the skin therefore include improvements that maintain and/or enhance the appearance of the skin such that it provides what is considered to be a youthful appearance, for example by repairing an ageing appearance. For example, visual properties of the skin that may be improved and/or enhanced include evening out skin tone, providing lift (preferably instant lift), giving skin a smooth, youthful appearance, re-plumping and replenishing skin (for example to provide immediate lift), re-texturing skin, recovering and brightening skin, reducing the appearance of dark spots, calming and reducing redness, improving clarity, improving radiance and/or improving colour.

References to improving the feel of the skin therefore include improvements that maintain and/or enhance feel of the skin such that it provides a smooth and even appearance, for example by re-plumping, re-texturing and/or softening the skin.

References to improving the skin by guarding against future problems associated with the skin of a subject may include protecting and/or moisturising the skin, for example by guarding against the premature visible ageing effects of sun.

In particular, the mannose-6-phosphate as a skin improvement agent improves skin colour, for example to provide and/or maintain a consistent colour across an area of the skin (such as across the face), i.e. the skin improvement is providing and/or maintaining a consistent skin colour.

In particular, the mannose-6-phosphate as a skin improvement agent calms and/or reduces redness, i.e. the skin improvement is reducing redness of the skin.

The skin improvement agent may improve and provide a cosmetic effect to skin that is considered normal, or more particularly to skin that has suffered any type of damage. When the cosmetic effect is provided to skin that is considered normal, then the improvement clearly provides merely a beautifying effect to the skin. By references to skin that has suffered damage we include damage caused by effects such as the ageing process and typical environmental and life style factors, as well as skin that has been subject to damage by any other means, including by means of a skin resurfacing procedure or caused by a skin disorder or condition.

The skin improvement agent as described herein is particularly useful for improving skin that has been subject to damage by means of a skin resurfacing procedure, which skin resurfacing procedure may be any such procedure including those described herein. The damage so caused may comprise damage to the epidermis alone or to both the epidermis and dermis. By damage to the epidermis we mean that at least a part of the epidermis has been removed from the skin of a subject, i.e. such that one or more (especially two or more) layers of the epidermis have been removed from the skin of the subject. By damage to the dermis, we mean that at least a part of the dermis has been removed from the skin of a subject, i.e. such that one or more layers of the dermis have been removed from the skin of the subject. As the skilled person would appreciate, when damage to the dermis occurs, then there is also damage to the epidermis, although damage may occur to the epidermis alone. In particular, the skin improvement agent as described herein is useful for improving skin wherein the damage has occurred at least to the stratum corneum and more particularly to the stratum corneum and at least one layer below the stratum corneum, for example so as to remove both dead and live cells from the skin. As the skilled person would appreciate, where reference is made to skin damaged by a skin resurfacing procedure, the damage occurs substantially to the area of the skin to which a skin resurfacing procedure has been applied.

In one aspect, the skin improvement agent as described herein is useful for improving skin that has been subject to damage, wherein the damage comprises the removal (or peeling off) of at least a portion of the epidermis, for example the removal (or peeling off) of the strataum corneum and at least one or more of the stratum lucidum, stratum granulosum, stratum spinosum and stratum basale. In the case where the strataum corneum, stratum lucidum, stratum granulosum and stratum spinosum and stratum basale are all removed, this represents the removal of the entire epidermis from an area of damaged skin and such damage may for example be provided by superficial skin resurfacing procedures.

In another aspect, the skin improvement agent as described herein is useful for improving skin that has been subject to damage, wherein the damage comprises the removal (or peeling off) of the entire epidermis of an area of damaged skin and of at least a portion of the dermis, especially of the upper reticular dermis. Such damage typically is provided by medium skin resurfacing procedures.

In another aspect, the skin improvement agent as described herein is useful for improving skin that has been subject to damage, wherein the damage comprises the removal (or peeling off) of the entire epidermis of an area of damaged skin and of at least a portion of the upper and mid-reticular dermis. Such damage typically is provided by deep skin resurfacing procedures.

As discussed above, the damage to the skin may have occurred by any means, as would be appreciated by a person skilled in the art. For example, the damage to the skin may result from a cosmetic skin resurfacing procedure that is conducted on a suitable area of skin. For example, the damage to the skin may result from the application of a chemical skin peel agent (especially a facial chemical skin peel agent) to the skin. Chemical skin peel agents are well known and are discussed above. The application of any such chemical skin peel agent to the skin may cause damage to the epidermis only or to both the epidermis and the dermis. In particular, the application of a medium or deep (especially deep) chemical skin peel agent to the skin, such as a medium peel comprising trichloroacetic acid (TCA) or a deep peel comprising phenol, may cause damage to the epidermis and the dermis. The application of a superficial chemical skin peel agent to the skin may cause damage to the epidermis only.

The damage to the skin may result from the application of laser peel treatment to the skin. Laser peel treatments are well known and are discussed above. The application of any such laser peel treatment to the skin may cause damage to the epidermis only, or to both the epidermis and the dermis. In particular, the application of a medium or deep (especially deep) laser peel treatment, such as treatment with a carbon dioxide and/or erbium: YAG laser, may cause damage to the epidermis and the dermis.

Alternatively, the damage to the skin may result from a split thickness skin graft. Split thickness skin grafting is the transplantation of a subject's own cutaneous tissue harvested from an area of normal skin, for example to replace an area of skin loss or injury. The damage caused by the graft is removal of the epidermis and damage to the underlying dermis.

The mannose-6-phosphate, pharmaceutically or cosmetically acceptable salt, precursor, or analogue thereof is eminently suitable for use in one or more of the following applications:
a. calming and/or reducing redness
b. easing the effects of sunburn
c. soothing nappy rash in babies
d. calming skin subject to shaving irritation
e. treating the undesirable cosmetic side-effects following a skin peel
f. soothing skin after exfoliation
g. a lip salve
h. dry skin preparations
i. mother and baby ointments
j. make up/foundation
k. anti ageing preparations

It is surprising that mannose-6-phosphate, and salts, precursors and analogues thereof, are capable of providing a cosmetic effect at the surface of normal and especially damaged skin. In particular, it is surprising that mannose-6-phosphate, and salts, precursors and analogues thereof, are capable of providing a cosmetic effect at the skin surface, for example by improving skin colour and/or tone so as to resemble more closely undamaged skin and in particular by reducing redness, particularly following damage thereto. As discussed above, mannose-6-phosphate has been shown to treat fibrotic disorders that would otherwise give rise to deleterious or pathological structures within the skin with the result of accelerating wound healing and/or preventing or mitigating scar formation in deep wounds, i.e. in full thickness wounds that include damage to the epidermis and through the complete dermal layer and subcutaneous fat. However, there has been no disclosure or suggestion that mannose-6-phosphate would improve the properties of the skin, especially improve skin colour and/or tone, more especially reduce redness, of the skin when applied to the surface of the skin in cases where no deleterious or pathological changes to the skin would be expected. Moreover, there has previously been no suggestion that mannose-6-phosphate would be suitable for use in a therapeutic method of treating skin redness.

According to the present invention, there is provided the use of mannose-6-phosphate, or a salt, precursor, or analogue thereof, for providing and/or maintaining a consistent skin colour, wherein the skin has been subject to a cosmetic skin resurfacing procedure.

According to the present invention, there is also provided a method for providing and/or maintaining a consistent colour of the skin of a subject, wherein the method comprises the steps of subjecting the skin to a cosmetic skin resurfacing procedure and administering an effective amount of mannose-6-phosphate, or a salt (especially a cosmetically acceptable salt), precursor, or analogue thereof, to the skin of the subject.

According to the present invention, there is provided the use of mannose-6-phosphate, or a salt (especially a cosmetically acceptable salt), precursor, or analogue thereof, for reducing redness of skin, wherein the skin has been subject to a cosmetic skin resurfacing procedure.

According to the present invention, there is also provided a method for reducing redness of the skin of a subject, wherein the method comprises the steps of subjecting the skin to a cosmetic skin resurfacing procedure and administering an effective amount of mannose-6-phosphate, or a salt (especially a cosmetically acceptable salt), precursor, or analogue thereof, to the skin of the subject.

According to the present invention, there is provided the use of mannose-6-phosphate, or a cosmetically acceptable salt, precursor, or analogue thereof, as a skin improvement agent for providing a cosmetic effect, wherein the skin has been subject to a cosmetic skin resurfacing procedure.

According to the present invention, there is also provided a method for providing a cosmetic effect by improving the skin of a subject, wherein the method comprises the steps of subjecting the skin to a cosmetic skin resurfacing procedure and administering an effective amount of mannose-6-phosphate, or a cosmetically acceptable salt, precursor, or analogue thereof, to the skin of the subject.

According to the present invention, there is also provided the use of mannose-6-phosphate, or a cosmetically acceptable salt, precursor, or analogue thereof, as a cosmetic product, wherein the skin has been subject to a cosmetic skin resurfacing procedure.

According to the present invention, there is also provided a method for providing a cosmetic effect to the skin of a subject, wherein the method comprises the steps of subjecting the skin to a cosmetic skin resurfacing procedure and administering an effective amount of mannose-6-phosphate, or a salt (especially a cosmetically acceptable salt), precursor, or analogue thereof, to the skin of the subject.

According to the present invention, there is also provided a method for providing a cosmetic effect to the skin of a subject, wherein the method comprises the steps of subjecting the skin to a cosmetic skin resurfacing procedure and administering an effective amount of a cosmetic composition, wherein the cosmetic composition comprises mannose-6-phosphate, or a cosmetically acceptable salt, precursor, or analogue thereof, to the skin of the subject.

According to the present invention, there is also provided the use of mannose-6-phosphate, or a cosmetically acceptable salt, precursor, or analogue thereof, as a skin improvement agent in a cosmetic composition, wherein the skin has been subject to a cosmetic skin resurfacing procedure.

According to the present invention, there is also provided a method for providing a cosmetic effect to the skin of a subject, wherein the method comprises the steps of subjecting the skin to a cosmetic skin resurfacing procedure and administering an effective amount of a cosmetic composition, wherein the cosmetic composition comprises mannose-6-phosphate, or a cosmetically acceptable salt, precursor, or analogue thereof, to the skin of the subject.

According to the present invention, there is also provided a cosmetic method for improving the skin of a subject, wherein the method comprises the steps of subjecting the skin to a cosmetic skin resurfacing procedure and administering an effective amount of mannose-6-phosphate, or a cosmetically acceptable salt, precursor, or analogue thereof, to the skin of the subject.

According to the present invention, there is further provided a cosmetic method for improving the skin of a subject, wherein the method comprises the steps of subjecting the skin to a cosmetic skin resurfacing procedure and administering an effective amount of mannose-6-phosphate, or a cosmetically acceptable salt, precursor, or analogue thereof, to the skin of the subject so as to provide a cosmetic effect to the skin.

As the skilled person would appreciate, the mannose-6-phosphate, or a cosmetically acceptable salt, precursor, or analogue thereof, is typically administered to substantially the same area of the skin that has been subjected to the cosmetic skin resurfacing procedure to provide optimum results.

According to the present invention, there is also provided a cosmetic method for improving the skin of a subject, wherein the method comprises the steps of administering an effective amount of a chemical skin peel agent (especially a facial chemical skin peel agent) to an area of the skin of the subject, administering an effective amount of mannose-6-phosphate, or a cosmetically acceptable salt, precursor, or analogue thereof, to substantially the same area of skin of the subject.

According to the present invention, there is further provided a cosmetic method for improving the skin of a subject, wherein the method comprises the steps of administering an effective amount of a chemical skin peel agent (especially a facial chemical skin peel agent) to an area of the skin of the subject, administering an effective amount of mannose-6-phosphate, or a cosmetically acceptable salt, precursor, or analogue thereof, to substantially the same area of the skin of the subject so as to provide a cosmetic effect to the skin.

According to the present invention, there is also provided a cosmetic method for improving the skin of a subject, wherein the method comprises the steps of subjecting an area of the skin to a laser peel treatment (especially a facial laser peel treatment), administering an effective amount of mannose-6-phosphate, or a cosmetically acceptable salt, precursor, or analogue thereof, to substantially the same area skin of the subject.

According to the present invention, there is further provided a cosmetic method for improving the skin of a subject, wherein the method comprises the steps of subjecting an area of the skin to a laser peel treatment (especially a facial laser peel treatment), administering an effective amount of mannose-6-phosphate, or a cosmetically acceptable salt, precursor, or analogue thereof, to substantially the same area of the skin of the subject so as to provide a cosmetic effect to the skin.

The mannose-6-phosphate, or cosmetically acceptable salt, precursor, or analogue thereof, may be administered to the skin before, after or simultaneously, with a skin resurfacing procedure. In particular, the mannose-6-phosphate, or cosmetically acceptable salt, precursor, or analogue thereof, may be administered to the skin after the skin resurfacing procedure (especially after a facial skin resurfacing procedure) has been conducted. For example, the mannose-6-phosphate, or cosmetically acceptable salt, precursor, or analogue thereof, may be administered to the skin after a chemical skin peel agent (especially a facial chemical skin peel agent) has been applied thereto, or after a skin laser treatment (especially a facial skin laser treatment) has been conducted. If the mannose-6-phosphate, or cosmetically acceptable salt, precursor, or analogue thereof, is administered to the skin before a skin resurfacing procedure, then the mannose-6-phosphate, or cosmetically acceptable salt, precursor, or analogue thereof, may conveniently be combined with a suitable sun cream or block.

In particular, the skin improvement agent improves skin colour, for example to provide and/or maintain a consistent colour across an area of the skin (such as across the face) so as to resemble more closely the colour of the skin before the damage occurred, i.e. the skin improvement provides and/or maintains a consistent skin colour. In particular, the skin improvement agent may reduce redness of the skin. Such an improvement in the skin colour and/or redness is particularly desirable when the skin has been treated with a skin resurfacing procedure (especially a facial skin resurfacing procedure) as described herein (particularly a medium or deep facial skin resurfacing procedure, especially a deep facial skin resurfacing procedure). As discussed above, a disadvantage associated with the use of skin resurfacing procedures is that they may cause areas of redness to the skin to which they are administered. This increases the time taken for a subject to fully recover from a skin resurfacing procedure, which time should be kept to a minimum with such a cosmetic treatment. Thus, the use of mannose-6-phosphate may reduce recovery times following a skin resurfacing procedure, especially following a medium or deep skin resurfacing procedure.

Thus, the uses and methods of the present invention are particularly intended to improve the colour of the skin, especially to reduce the redness of skin, which skin has been subjected to a skin resurfacing procedure. The uses and methods may reduce the contrast in colour of the skin, especially the redness, in comparison to skin that has not been damaged by a skin resurfacing procedure. Conveniently, uses and methods may reduce the contrast in colour of the skin, especially the redness, so as to provide an improvement over skin prior to its being damaged by a skin resurfacing procedure. The reduction in redness and/or contrast is measurable. It is believed that the reduction in redness provided by mannose-6-phosphate in the present invention may be due to an acceleration of the processes by which redness is reduced in the absence of treatment with a suitable agent, for example so as to reduce the time that it takes the colour (especially the redness) of the skin to return to normal after damage.

For example, the reduction in redness and/or contrast may be measured visually by means of a photographic assessment. Any suitable assessor may perform the photographic assessment of treated and untreated skin. Examples of suitable assessors include independent lay or expert panels, clinicians, or the subjects themselves. Treated or untreated skin may be assessed compared to standardised and calibrated photographs of skin. An advantageous application of photographic assessment of skin lies in the assessment of skin colour, and in particular skin redness. This may preferably be carried out by comparison of standardised clinical photographs (for example photographs in which the parameters influencing colour are controlled from photograph to photograph). Suitable procedures by which images may be standardised and calibrated for such applications are well known to those skilled in the art.

In a preferred method, standardised images of skin (such as treated and non-treated skin, or treated skin at various time-points preceding, during and/or after treatment) may be loaded into any image analysis or suitable digital photo editing software, such as Adobe Photoshop, and the outline of the skin (or areas of skin to be analysed) traced. Global colour analysis measurements (ΔL, Δa and Δb) may then be calculated as the difference in L (lightness), a (red/green value) and b (yellow/blue) values between the area inside the traced outline (the skin area) and outside the traced line (the surrounding skin area). Comparison of the overall colour difference (ΔE) allows a quantitative assessment of skin colour to be made. This colour can be compared directly with the surrounding untreated skin and/or placebo treated skin or compared as the difference between the comparisons of untreated to treated skin versus the comparison of untreated to placebo treated skin. These colour analysis measurements may be used to provide information regarding skin redness, a reduction in which is indicated by a shift in Δa value between the treated skin pre and post treatment. The reduction in redness and/or colour/contrast of the skin therefore is quantifiable by means of a difference in ΔL, Δa, Δb and/or ΔE.

The present invention further provides the use of mannose-6-phosphate, or a cosmetically acceptable salt, precursor, or analogue thereof, in the manufacture of a cosmetic composition. There is also provided the use of mannose-6-phosphate, or a cosmetically acceptable salt, precursor, or analogue thereof, in the manufacture of a cosmetic composition for use in providing and/or maintaining a consistent skin colour of a subject. There is also provided the use of mannose-6-phosphate, or a cosmetically acceptable salt, precursor, or analogue thereof, in the manufacture of a cosmetic composition for use in reducing the redness of the skin of a subject. There is also provided the use of mannose-6-phosphate, or a cosmetically acceptable salt, precursor, or analogue thereof, in the manufacture of a cosmetic composition for use in providing a cosmetic effect, particularly for use in improving the skin of a subject. There is also provided the use of mannose-6-phosphate, or a cosmetically acceptable salt, precursor, or analogue thereof, as a skin improvement agent in the manufacture of a cosmetic composition for use in providing a cosmetic effect, particularly for use in improving the skin of a subject.

The mannose-6-phosphate, or a cosmetically acceptable salt, precursor, or analogue thereof, may be used in any of the uses and methods described herein in any suitable form. Typically, the mannose-6-phosphate, or a cosmetically acceptable salt, precursor, or analogue thereof, is formulated as a cosmetic composition.

Thus, the present invention further provides a cosmetic composition comprising mannose-6-phosphate, or a cosmetically acceptable salt, precursor, or analogue thereof, and a cosmetically acceptable carrier.

By a “cosmetic composition” we mean a composition including mannose-6-phosphate, or a cosmetically acceptable salt, precursor, or analogue thereof, that is suitable for administration to a subject (especially to the skin of the subject) so as to provide a cosmetic effect. In particular, the cosmetic composition may be suitable for administration to the face, neck and/or hand, especially to the face and/or neck, more especially to the face, of the subject.

The cosmetic composition of the present invention comprises mannose-6-phosphate as, or a cosmetically acceptable salt, precursor, or analogue thereof, a skin improvement agent, such that when the composition is administered to the skin of a subject (especially to the face, neck and/or hand, more especially to the face and/or neck, even more especially to the face, of the subject) it improves the skin, particularly the macroscopic properties of the skin, more particularly reduces redness of the skin.

Typically, the cosmetic composition is for direct administration to the skin of a subject. For example, the cosmetic composition typically is for administration to the skin (of the subject) by topical administration. By topical administration we mean delivery of the skin improvement agent by application to the skin by any suitable means.

Any suitable cosmetically acceptable carrier may be included in the cosmetic compositions of the invention, which carriers typically would be well known to persons skilled in the art. The particular carriers selected depend on various factors, including the mode of administration of the composition and the stability of the mannose-6-phosphate. The cosmetically acceptable carrier should be selected so as to deliver a sufficient amount of the mannose-6-phosphate to the skin.

The cosmetic compositions may be made in a wide range of product types that include, but are not limited to, solid and liquid compositions such as lotions, creams, gels, sticks, sprays, shaving creams, ointments, cleansing liquid washes, scrubs and solid bars, shampoos, pastes, powders, mousses, waxes, serums, masks, adhesive strips, patches, films and wipes. These product types may comprise several types of cosmetically acceptable carriers including solutions, emulsions (for example microemulsions and nanoemulsions), gels, solids and liposomes. Other carriers may be formulated by those skilled in the art. Films may be dissolvable absorbable films that may be placed on the skin and then dissolve. Such films may, for example, be comprised of pectin, collagen, gelatin, hydroxypropylmethylcellulose, hydroxypropylcellulose, and/or modified carboxymethylcellulose.

Cosmetic compositions for topical administration may for example be in the form of solutions, creams, ointments, jellies, gels, sprays, foams, powders, waxes, liposomes, emulsions (for example microemulsions and nanoemulsions), or aqueous or oily solutions or suspensions (especially in the form of powders, solutions or suspensions). Suitable carriers include, for example, water and water-miscible liquids such as alcohols (for example ethanol, propanol or butanol), glycols (for example ethylene glycol, dipropylene glycol or butylene glycol), silicone oils, vegetable oils, and mixtures of water miscible liquids and water.

Cosmetic compositions for topical administration by application to the skin may include moisturisers, and sun tan lotions and creams.

The mannose-6-phosphate, salt, precursor, or analogue thereof is present in the cosmetic composition in a cosmetically effective amount. A “cosmetically effective amount” is any amount of mannose-6-phosphate which, when administered to a subject causes improvement to the skin and in particular improves the colour, for example reduces redness, of the skin. For example, a suitable cosmetically effective amount of mannose-6-phosphate, salt, precursor, or analogue thereof in a composition for topical administration may be in the region of about 0.01 to 50% by weight, more preferably around 1 to 50%, or for example about 1 to 20%. More preferably the mannose-6-phosphate, salt, precursor, or analogue thereof may be present in a cosmetically effective amount of around 1 to 5% by weight, still more preferably around 1 to 3% by weight. In some applications it may be preferred to use higher levels of mannose-6-phosphate, salt, precursor, or analogue thereof, such as about 10 to 20% by weight. For example, a cosmetic composition comprising mannose-6-phosphate, its salt, precursor, or analogue thereof may comprise greater than 9% by weight, for example a range of greater than 9 to 50% by weight mannose-6-phosphate, salt, precursor, or analogue thereof. Cosmetic compositions comprising greater than 9% by weight mannose-6-phosphate, salt, precursor, or analogue thereof may provide particular advantages in terms of skin improvements when administered to skin that has been subject to a deep skin resurfacing procedure.

Topical administration may be achieved by means of diffusion from or through a suitable material to the skin, i.e. wherein the mannose-6-phosphate is releasably contained in or applied to the material for release to the skin upon contact therewith. Examples of such suitable materials include bandages, plasters, masks and films. For example, the mannose-6-phosphate may be provided in a mask, such as in a facial gel mask for effective administration to the face.

There is further provided a cosmetic composition comprising mannose-6-phosphate, or a cosmetically acceptable salt, precursor, or analogue thereof, and a cosmetically acceptable carrier, wherein the carrier comprises an aqueous medium.

There is further provided a cosmetic composition comprising mannose-6-phosphate, or a cosmetically acceptable salt, precursor, or analogue thereof, and a cosmetically acceptable carrier, wherein the composition comprises about 0.01 to 50% by weight of the mannose-6-phosphate, or cosmetically acceptable salt, precursor or analogue thereof, more preferably around 1 to 50%, or for example about 1 to 20%. More preferably the mannose-6-phosphate, salt, precursor, or analogue thereof may be present in the composition in an amount of around 1 to 5% by weight, still more preferably around 1 to 3% by weight. In some applications it may be preferred to use higher levels of mannose-6-phosphate, salt, precursor, or analogue thereof, such as about 10 to 20% by weight. There is further provided a cosmetic composition comprising mannose-6-phosphate, or a cosmetically acceptable salt, precursor, or analogue thereof, and a cosmetically acceptable carrier, wherein the carrier comprises greater than 9% by weight, for example a range of greater than 9% to 50% by weight of the mannose-6-phosphate, or cosmetically acceptable salt, precursor, or analogue thereof.

There is further provided a cosmetic composition comprising a cosmetically acceptable salt of mannose-6-phosphate and a cosmetically acceptable carrier. In particular, the salt may be any suitable salt other than a sodium salt.

There is further provided a cosmetic composition comprising a precursor of mannose-6-phosphate and a cosmetically acceptable carrier.

There is further provided a cosmetic composition comprising an analogue of mannose-6-phosphate and a cosmetically acceptable carrier.

Cosmetic compositions of the present invention may include any suitable additional ingredients, such as diluents, colourants, cosmetic additives, preservatives, fragrances, emollients, emulsifiers, anti-oxidants, anti-ageing agent, anti-wrinkle agents, collagen promoters, radiance enhancers/opacifiers, UV filters, Sun Protection Factor (SPF) agents, cleansing agents, anti-irritant agents, anti-bacterial agents, thickeners, buffers and moisturising agents, and combinations thereof.

Anti-bacterial agents may advantageously be included in the cosmetic compositions of the present invention. Suitable anti-bacterial agents include, for example, methylparaben, propylparaben, butylparaben, ethylparaben, isobutylparaben, benzyl alcohol, DMDM hydantoin (i.e. 1,3-dimethylol-5,5-dimethylhydantoin), silver citrate, triclosan, benzalkonium chloride and phenoxyethanol, and combinations thereof.

The present invention further provides a chemical skin peel composition comprising mannose-6-phosphate, or a cosmetically acceptable salt, precursor, or analogue thereof, and an active chemical skin peel ingredient. The present invention further provides a chemical facial skin peel composition comprising mannose-6-phosphate, or a cosmetically acceptable salt, precursor, or analogue thereof, and an active chemical facial skin peel ingredient. The active chemical skin (especially facial) peel ingredient may be any known such ingredient, for example trichloroacetic acid or phenol. Preferably, when a salt of mannose-6-phosphate is included in a chemical skin peel composition, the salt is not a sodium salt.

The cosmetic compositions of the invention may be obtained by conventional procedures using conventional cosmetic carriers, well known in the art.

The cosmetic compositions of the invention have beneficial cosmetic properties and may be provided for any of the uses and methods as described herein. The cosmetic compositions of the invention may be provided for the improvement of the skin, especially for improving the colour, for example reducing redness, of the skin, of a subject as discussed herein.

There is also provided a kit comprising mannose-6-phosphate, or a cosmetically acceptable salt, precursor, or analogue thereof, and a dispenser for the mannose-6-phosphate, or the cosmetically acceptable salt, precursor, or analogue thereof. The kit may further provide instructions for administering the mannose-6-phosphate, for example for the topical administration of the mannose-6-phosphate. Any suitable dispenser may be used, as would be appreciated by persons skilled in the art. The mannose-6-phosphate, or a cosmetically acceptable salt, precursor, or analogue thereof, may be provided in the kit in the form of a cosmetic composition as described herein.

It is clear from the method of action of a skin resurfacing procedure (especially a facial skin resurfacing procedure) as discussed above, which burns the skin upon application thereto, that these procedures may cause some damage to the skin of a medical nature, as well as of a cosmetic nature. For example, the burning of the skin causes damage to the skin of a medical nature that may require medical treatment or intervention, as well as damage of a cosmetic nature as discussed above. The treatment of medical skin damage, such as burns and associated conditions/disorders, may also be desirable and in another aspect of the present invention may be addressed. Such a medical treatment may be of a prophylactic or curative type.

The medical treatment may be provided to skin damage at any area of the body of the subject (especially to the face, neck and/or hand, especially to the face and/or neck, more especially to the face, of the subject).

For example, a medical treatment may comprise a reduction of the likelihood of infection, i.e. may inhibit the growth of bacteria in damaged or burnt skin, for example in skin that has been damaged or burnt by means of a procedure such as a skin resurfacing procedure. This in turn may reduce recovery time. A medical treatment may also comprise a reduction in swelling at the site of damage.

Thus, the present invention further provides mannose-6-phosphate, or a pharmaceutically acceptable salt, precursor, or analogue thereof, for use in treating skin that has been subject to damage, especially to damage to the epidermis or to both the epidermis and dermis. This aspect of the present invention relates to a medical method of treatment, for example to treat damage caused to the skin by any means. Treatment may include reducing swelling of the skin. Treatment may also comprise the inhibition of infection, especially when mannose-6-phosphate is administered with an anti-bacterial agent. Treatment may also comprise reducing redness of the skin.

There is also provided the use of mannose-6-phosphate, or a pharmaceutically acceptable salt, precursor, or analogue thereof, in the manufacture of a medicament for use in the treatment of skin that has been subject to damage (especially to damage to the epidermis or to both the epidermis and dermis).

There is also provided a method for the treatment of skin that has been subject to damage (especially to damage to the epidermis or to both the epidermis and dermis), wherein the method comprises the step of administering mannose-6-phosphate, or a pharmaceutically acceptable salt, precursor, or analogue thereof, to the damaged skin of the subject.

As a medical use of the present invention there may be provided mannose-6-phosphate, or a pharmaceutically acceptable salt, precursor, or analogue thereof, for reducing redness of damaged skin.

There is also provided the use of mannose-6-phosphate, or a pharmaceutically acceptable salt, precursor, or analogue thereof, in the manufacture of a medicament for use in reducing redness of damaged skin.

There is also provided a method for reducing redness of damaged skin, wherein the method comprises the step of administering mannose-6-phosphate, or a pharmaceutically acceptable salt, precursor, or analogue thereof, to the damaged skin of the subject.

There may also be provided mannose-6-phosphate, or a pharmaceutically acceptable salt, precursor, or analogue thereof, for inhibiting the growth of bacteria on damaged skin, i.e. for use as a bacteriostatic agent.

There is also provided the use of mannose-6-phosphate, or a pharmaceutically acceptable salt, precursor, or analogue thereof, in the manufacture of a medicament for inhibiting the growth of bacteria on damaged skin.

There is also provided a method for inhibiting the growth of bacteria on damaged skin, wherein the method comprises the step of administering mannose-6-phosphate, or a pharmaceutically acceptable salt, precursor, or analogue thereof, to the damaged skin of the subject.

There may also be provided mannose-6-phosphate, or a pharmaceutically acceptable salt, precursor, or analogue thereof, for reducing swelling of damaged skin.

There is also provided the use of mannose-6-phosphate, or a pharmaceutically acceptable salt, precursor, or analogue thereof, in the manufacture of a medicament for use in reducing swelling of damaged skin.

There is also provided a method for reducing swelling of damaged skin, wherein the method comprises the step of administering mannose-6-phosphate, or a pharmaceutically acceptable salt, precursor, or analogue thereof, to the damaged skin of the subject.

There may also be provided the use of mannose-6-phosphate, or a salt, precursor, or analogue thereof, for providing and/or maintaining a consistent skin colour of damaged skin. For example skin that it is desirable to treat so as to provide a consistent skin colour may be damaged by means of a medical condition, such as melasma, erythrasma, pityriasis alba, rash, vitiligo, rosacea, port wine stain, stretch marks or any disorder which has a symptom of erythema, or damage caused by means of radiation therapy.

There is also provided the use of mannose-6-phosphate, or a pharmaceutically acceptable salt, precursor, or analogue thereof, in the manufacture of a medicament for providing and/or maintaining a consistent skin colour of damaged skin.

There is also provided a method for providing and/or maintaining a consistent skin colour of damaged skin, wherein the method comprises the step of administering mannose-6-phosphate, or a pharmaceutically acceptable salt, precursor, or analogue thereof, to the damaged skin of the subject.

In the medical methods of treatment and the medical uses discussed above, the damage to the skin may have occurred by any means, as would be appreciated by a person skilled in the art. Examples of such means by which such damage may occur are discussed above in relation to the cosmetic methods and uses. For example, the damage may result from a skin resurfacing procedure, as discussed herein. For example, the damage may result from the application of a chemical skin peel product (especially a chemical facial skin peel product) to the skin, i.e. the application of which chemical skin peel product produces a burn on the skin requiring medical treatment. In particular, the application of a medium or deep (especially deep) chemical facial skin peel product to the skin may cause damage in the form of such a burn. For example, the damage may result from the application of a laser treatment, as discussed herein.

The mannose-6-phosphate may be used in any of the medical uses and methods described herein in any suitable form. Typically, the mannose-6-phosphate is formulated as a pharmaceutical composition.

By a “pharmaceutical composition” we mean a composition including an active pharmaceutical ingredient that is suitable for administration to a subject so as to treat and/or prevent a medical disorder/condition, i.e. so as to provide a therapeutic effect. An active pharmaceutical ingredient is an agent that is effective against a disorder/condition, such that when it is administered to a subject suffering from the disorder/condition it causes reduction, remission, or regression of the disorder/condition. An active pharmaceutical ingredient is also typically effective in the prevention of a disorder/condition upon administration to a subject, i.e. so as to prevent and/or delay onset of the disorder/condition.

The pharmaceutical compositions of this aspect of the present invention comprise mannose-6-phosphate, or a pharmaceutically acceptable salt, precursor, or analogue thereof, as the active ingredient. Thus, the pharmaceutical compositions of the present invention comprise mannose-6-phosphate for administration to a subject (especially to the skin of the subject) so as to provide a pharmaceutical effect. In particular, the pharmaceutical compositions may be suitable for administration to the face, neck and/or hand, especially the face and/or neck, more especially the face, of the subject.

Typically, the pharmaceutical composition is for direct application to the skin of a subject. For example, the pharmaceutical composition may be for application to the skin (of the subject) by topical administration.

Any suitable pharmaceutically acceptable carrier may be included in the pharmaceutical compositions of the invention, which carriers typically would be well known to persons skilled in the art. The particular carriers selected depend on various factors, including the mode of administration of the composition and the stability of the mannose-6-phosphate. The pharmaceutically acceptable carrier should be selected so as to deliver a sufficient amount of the mannose-6-phosphate to the skin.

Suitable pharmaceutical carriers include inert solid diluents or fillers, sterile aqueous solutions and various organic solvents, as would be well known to persons skilled in the art.

The pharmaceutical compositions of the invention may be formulated for administration by any convenient route. For example, the pharmaceutical compositions may be in a form suitable for topical administration (for example as creams, ointments, gels, or aqueous or oily solutions or suspensions, or by transdermal delivery).

The mannose-6-phosphate, salt, precursor, or analogue thereof is present in the pharmaceutical composition in a therapeutically effective amount. A “therapeutically effective amount” is any amount of mannose-6-phosphate, salt, precursor, or analogue thereof (for example as contained in the pharmaceutical composition as described herein) which, when administered to a subject suffering from a disorder against which it is effective, causes prevention, reduction, remission and/or regression of the disorder.

The therapeutically effective amount of mannose-6-phosphate, salt, precursor, or analogue thereof that is combined with the pharmaceutically acceptable carrier to produce a single dosage form will necessarily vary depending upon the nature and severity of the disorder treated, the particular patient treated and the particular route of administration, according to well known principles of medicine. While the mannose-6-phosphate, salt, precursor, or analogue thereof may be administered at an appropriate level to a patient group subject to pre-existing medical conditions including fibrotic disorders, such as those discussed above which have given rise to deleterious or pathological structures within the skin, the mannose-6-phosphate, salt, precursor, or analogue thereof may be administered to patient groups which are not currently suffering from such fibrotic disorders but which have skin conditions such that the patients exhibit an undesirable cosmetic appearance, such as redness. Typically, compositions for topical administration (such as a cream) will contain about 0.01 to 50% of the mannose-6-phosphate, or pharmaceutically acceptable salt, precursor or analogue thereof, more preferably around 1 to 50%, or for example about 1 to 20%. More preferably the mannose-6-phosphate, salt, precursor, or analogue thereof may be present in a pharmaceutically effective amount of around 1 to 5% by weight, still more preferably around 1 to 3% by weight. In some applications it may be preferred to use higher levels of mannose-6-phosphate, salt, precursor, or analogue thereof, such as about 10 to 20% by weight. For example, a pharmaceutical composition comprising mannose-6-phosphate, salt, precursor, or analogue thereof may comprise greater than 9% by weight, for example a range of greater than 9% to 50% by weight mannose-6-phosphate, its salt, precursor, or analogue thereof. Pharmaceutical compositions comprising greater than 9% by weight mannose-6-phosphate, its salt, precursor, or analogue thereof may be particularly useful for treating skin that has been subject to deep skin resurfacing procedures.

The pharmaceutical compositions of the invention may be obtained by conventional procedures using conventional pharmaceutical carriers, well known in the art. The pharmaceutical composition of the invention may be provided for any of the medical methods and uses as described herein.

The structure of mannose-6-phosphate would be well known to persons skilled in the art. References herein to mannose-6-phosphate, and salts, precursors and analogues thereof, include all stereoisomers (including enantiomers, diastereomers (including anomers) and geometric isomers) and mixtures (including racemic and scalemic mixtures) thereof. Any stereoisomer or mixture of stereoisomers of mannose-6-phosphate, and salts, precursors and analogues thereof, may be used in the present invention.

In particular, the mannose-6-phosphate used as described herein is the natural D-isomer, which has the structure:

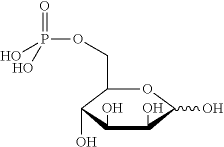

References herein to “mannose-6-phosphate” include cosmetically and pharmaceutically acceptable salts, precursors and analogues thereof as discussed herein, unless otherwise stated.

In an aspect of the invention, mannose-6-phosphate, especially D-mannose-6-phosphate, may be administered in the form of the free phosphoric acid.

GMP (pharmaceutically pure) mannose-6-phosphate may be used in the present invention as described herein.

Cosmetically acceptable salts of mannose-6-phosphate may be used in the cosmetic applications discussed herein and may be monovalent (i.e. in a 1:1 or 2:1 stoichiometry), divalent, or mixed monovalent (i.e. different monovalent salts). Suitable cosmetically acceptable salts include mono- or di- alkali or alkaline earth metal salts, for example sodium, potassium, lithium, calcium, magnesium, aluminium, zinc or barium salts, and organic amine salts, such as ammonium salts. Other suitable organic amine salts include long chain primary, secondary, tertiary and quaternary amines, for example decyl-, lauryl-, myristyl-, palmityl- or stearyl-amine. Also, shorter chain primary, secondary, tertiary and quaternary amine salts may be used. Examples of primary shorter chain amines include 2-aminoethanol, 2-aminoheptane, 2-amino-2-methyl- 1,3 propanediol, 2-amino-2-methyl-1-propanol, n-amylamine, benzylamine, 1,4-butanediamine, n- butylamine, cyclohexylamine, ethylamine, ethylenediamine, methylamine, a-methylbenzylamine, phenethylamine, propylamine, and tris (hydroxymethyl) aminomethane. Examples of shorter chain secondary amines include diethanolamine, diethylamine, diisopropylamine, and dimethylamine. Examples of shorter chain tertiary amines include N,N-diethylaniline, N,N-dimethylglycine, triethanolamine, triethylamine, and trimethylamine. Amines may also be used to form crystalline salts with mannose-6-phosphate, for example dicyclohexylamine, piperazine, benzylhydrylamine, amantadine, or tris(hydroxymethyl)aminomethane. Phosphonium salts may also be formed due to their different solubility and lipophilicity.

Pharmaceutically acceptable salts of mannose-6-phosphate may be used in the medical/pharmaceutical applications discussed herein. Suitable pharmaceutically acceptable salts are as discussed above for cosmetically acceptable salts and include mono- or di- alkali or alkaline earth metal salts, for example sodium., potassium lithium, calcium, magnesium, aluminium, zinc or barium salts, and organic amine salts, such as ammonium. Other pharmaceutically acceptable amine salts include N-methylglucamine, cholinate (N,N,N-trimethyl ethanolamine hydroxide) and tromethamine. In addition to these, other pharmaceutically suitable salts may be used which will be obvious to persons skilled in the art, such as described in ‘Pharmaceutical Salts: Properties, Selection, and Use’; Stahl, P. Heinrich/Wermuth, Camille G (eds. Wiley-V C H)

By precursors of mannose-6-phosphate, we mean any precursor form of mannose-6-phosphate that is suitable for administration to a subject that releases mannose-6-phosphate upon administration to the area of the skin requiring improvement or treatment. For example, such a precursor may be a compound that is converted to mannose-6-phosphate in situ upon administration to the body (for example skin) of the subject. Suitable such precursors may for example comprise esters of mannose-6-phosphate, formed for example by the reaction of a sugar alcohol group with an appropriate acid, such that the ester group is hydrolysed upon administration to the subject to release the mannose-6-phosphate. Alternatively, the precursor may be another compound, such as an amide or carbonate, that releases mannose-6-phosphate by a hydrolytic, enzymatic or other reaction upon administration of the compound to the subject. The precursor may further be any polymeric form of mannose-6-phosphate, for example comprising at least one and preferably two or more mannose-6-phosphate monomers. In a di- or poly-saccharide, mannose-6-phosphate residues may be 1,4- or 1,6-linked to neighbouring mannose-6-phosphate or other saccharide residues. The polymers may comprise up to 20, particularly up to 10, mannose-6-phosphate monomers or may include mannose-6-phosphate as residues pendant from a backbone of a suitable polymer.

By analogues of mannose-6-phosphate, we mean any structurally related compound to mannose-6-phosphate that is suitable for administration to a subject and that provides the same cosmetic or therapeutic effect as mannose-6-phosphate and as described herein. For example, such an analogue may be a phosphonate analogue of mannose-6-phosphate, or a cosmetically or pharmaceutically acceptable salt of a phosphonate analogue. As the skilled person would appreciate, by a phosphonate analogue we mean a compound containing the mannose core but wherein the phosphate group of mannose-6-phosphate has been replaced by a phosphonate group, i.e. including a phosphonate group at the 6-position on the mannose core. Examples of suitable phosphonate analogues are described in WO-97/05883 and may include 2-(3,4,5,6-tetrahydroxytetrahydro-2H-pyran-2-yl)ethylphosphonic acid, 1,1-difluoro-2-(3,4,5,6-tetrahydroxytetrahydro-2H-pyran-2-yl)ethylphosphonic acid, hydroxyl(3,4,5,6-tetrahydroxytetrahydro-2H-pyran-2-yl)methylphosphonic acid, 2-(3,4,5,6-tetrahydroxytetrahydro-2H-pyran-2-yl)vinylphosphonic acid and 1-fluoro-2-(3,4,5,6-tetrahydroxytetrahydro-2H-pyran-2-yl)vinylphosphonic acid, having the following structures:

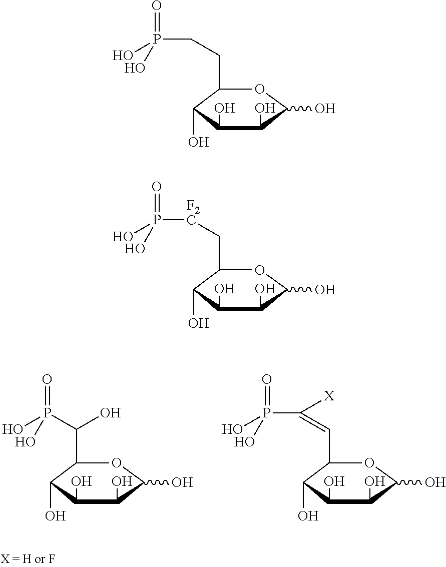

As the skilled person would appreciate, mannose-6-phosphate may be administered in the form of the free phosphoric acid or in the form of a salt, precursor or analogue thereof, or a combination of two or more such forms may be administered together.

While any suitable cosmetically or pharmaceutically acceptable salt, precursor or analogue of mannose-6-phosphate may be used in one or more of the aspects of the present invention defined herein it may be preferable to use cosmetically or pharmaceutically pure mannose-6-phosphate, i.e. mannose-6-phosphate which is sufficiently free of related salts, precursors or analogues so as to be considered ‘pure’ mannose-6-phosphate from a cosmetic or pharmaceutical perspective.

Mannose-6-phosphate, cosmetically and pharmaceutically acceptable salts, precursors and analogues thereof may be prepared by any suitable method and may be commercially available, as would be appreciated by persons skilled in the art. For example, phosphonate analogues of mannose-6-phosphate may be prepared as described in WO-97/05883.

Stereoisomers may be separated using conventional techniques, for example chromatography or fractional crystallisation. The enantiomers may be isolated by separation of a racemate for example by fractional crystallisation, resolution or HPLC. The diastereoisomers may be isolated by separation by virtue of the different physical properties of the diastereoisomers, for example, by fractional crystallisation, HPLC or flash chromatography. Alternatively particular stereoisomers may be made by chiral synthesis from chiral starting materials under conditions that will not cause racemisation or epimerisation, or by derivatisation, with a chiral reagent.

Following the topical administration of mannose-6-phosphate to the skin, for either a cosmetic or medical use, the mannose-6-phosphate may optionally be rinsed from the skin after a set time. Any suitable rinse agent may be used, for example water (especially sterilised water). The set time before rinsing will depend on several factors, including, in the case of treatment following damage, the severity of the damage.

References herein to a “subject” are intended to include mammals and birds. In particular, the subject is a warm-blooded animal, such as a domestic animal or man, particularly man.

References herein to administering mannose-6-phosphate to the skin of a subject refer to administration to any desired area of the skin, including but not limited to the face, neck, hand, leg and back. Typically, administration may be to the face, neck and/or hand, particularly to the face and/or neck, more particularly to the face.

### EXAMPLE

The ability of mannose-6-phosphate (also referred to as “M6P”) to bring about the effective improvement of skin properties was demonstrated using a clinical study/trial as set out below. A double blind, placebo controlled, within subject trial was conducted to investigate the efficacy and safety of mannose-6-phosphate to improve skin that had been subject to epithelial and superficial dermal damage. In the study/trial set out below, the skin damage resulted from a split thickness skin graft. This graft removes a partial layer of the skin and is considered to model the skin damage produced by a deep skin resurfacing procedure.

Mannose-6-phosphate was formulated as a solution by dissolving the required amount of mannose-6-phosphate in phosphate buffered saline at a pH of 6.5 to 7.5 and administered topically to volunteer subjects.

Prior to evaluation in humans a series of regulatory (Good Laboratory Practice; GLP) compliant preclinical safety studies had been conducted in relation to medical use indications to show that mannose-6-phosphate was safe to administer to humans.

The Figures show the following:

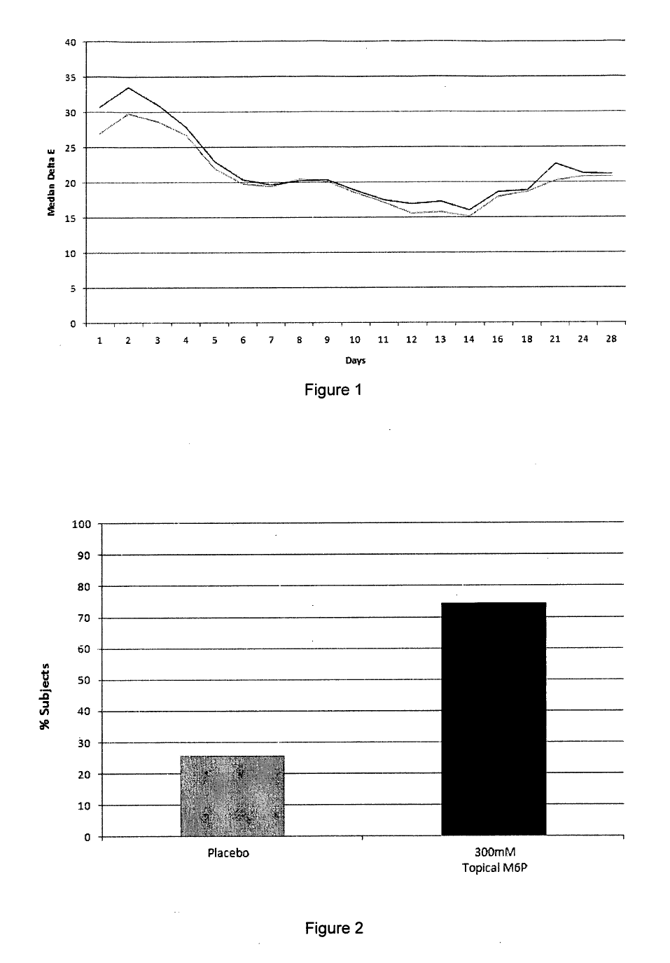

FIG. 1: Skin appearance as measured by median ΔE. The dark line represents data from placebo treated graft sites; the light line represents data from M6P treated graft sites (300 mM). A lower value represents an improvement in appearance. p=0.0045.

FIG. 2: Percentage of subjects with reduced redness between within-subject matched topical placebo or M6P (300mM) treated graft sites, as measured by Δa.

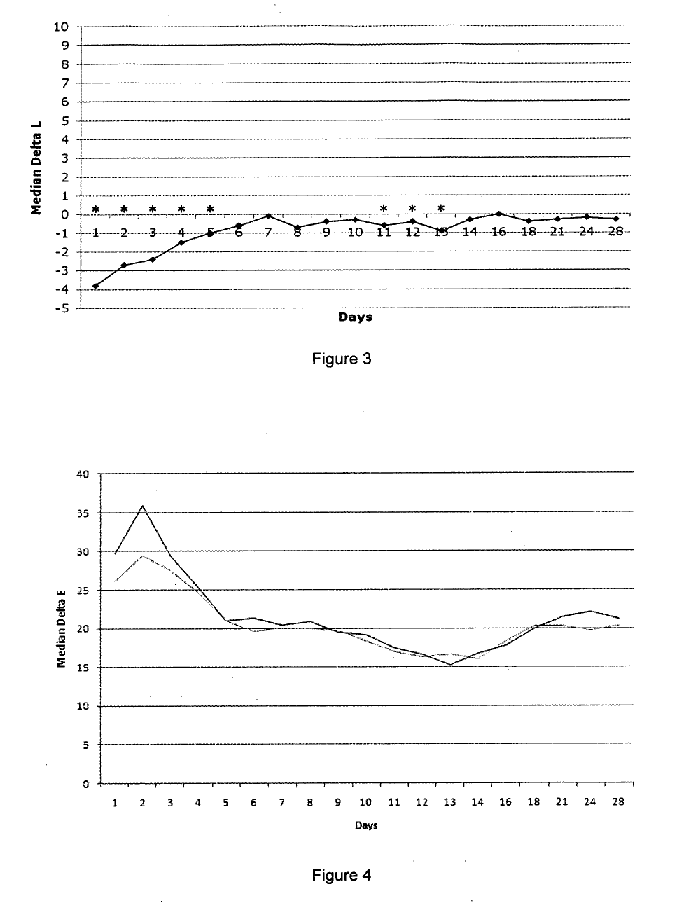

FIG. 3: Skin darkness/lightness as measured by median ΔL (placebo minus M6P values; a value below 0 on the y axis indicates M6P treated graft sites (300 mM) are lighter and blend better with the surrounding skin); * p<0.05.

FIG. 4: Skin appearance as measured by median ΔE. The dark line represents data from placebo treated graft sites; the light line represents data from M6P treated graft sites (600 mM). A lower value represents an improved appearance. p=0.0003.

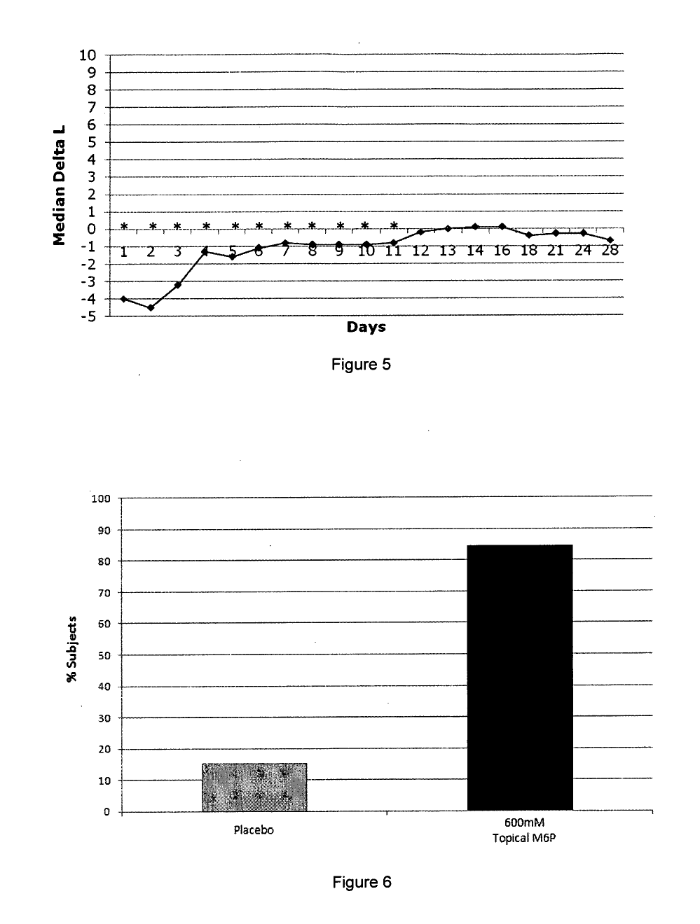

FIG. 5: Skin darkness/lightness as measured by median ΔL (placebo minus M6P values; a value below 0 on the y axis indicates M6P treated graft sites (600 mM) are lighter and blend better with the surrounding skin); *p<0.05.

FIG. 6: Percentage of subjects with reduced redness between within-subject matched topical placebo or M6P (600 mM) treated graft sites, as measured by Δa.

The trial was a single-centre (UK), double-blind, placebo-controlled, within-patient controlled, randomised trial performed in 195 healthy male and female subjects aged 18 to 70. Each subject had two 1.5 cm×2 cm graft donor sites marked on their lower back and were randomised to one of five treatment groups in the trial. The data from the groups treated with 300 mM or 600 mM M6P by topical application within 30 minutes of grafting and 24 hours later, versus within-subject placebo control, are reported here. Subjects attended visits over the 28-day study period. Photographs of the graft sites were taken throughout the study and quantitative measures of skin appearance, skin redness and skin darkness/lightness (ΔE, Δa and ΔL respectively) were made from standardised photographs (i.e. derived from the analysis of standardized photographs using CIElab (D65, 10°. The study protocol was granted ethical approval by an independent ethics and regulatory committee and the UK regulatory authority (Medicines and Healthcare Products Regulatory Agency [MHRA]). All subjects provided written informed consent prior to study commencement.

As discussed herein, ΔE is a combined quantitative measurement of ΔL (lightness/darkness axis), Δa (red/green axis) and Δb (blue/yellow axis). ΔL is the quantitative measurement of lightness/darkness axis. Δa is the quantitative measurement of red/green axis. Δb is the quantitative measurement of blue/yellow axis.

The CIE (Comission Internationale de l'Eclairage) Colour Model would be well known to persons skilled in the art. This model is completely independent of any device or other means of emission or reproduction and is based as closely as possible on how humans perceive colour. For the avoidance of doubt, the key elements of the CIE model are the definitions of standard sources and the specifications for a standard observer. CIELAB distinguishes between light and dark, red and green, and blue and yellow and indicates these values with three axes: L*, a*, and b*, as shown below:

The central vertical axis represents lightness (signified as L*) whose values run from 0 (black) to 100 (white). The colour axes are based on the fact that a colour cannot be both red and green, or both blue and yellow, because these colors oppose each other. On each axis the values run from positive to negative. On the a-a′ axis, positive values indicate amounts of red while negative values indicate amounts of green. On the b-b′ axis, yellow is positive and blue is negative. For both axes, zero is neutral grey. Therefore, values are only needed for two colour axes and for the lightness or greyscale axis (L*), which is separate.

Evaluation of the effects of M6P applied topically (once at the time of skin damage and once 24 hours later) surprisingly demonstrated a number of beneficial effects at the 300 mM and 600 mM doses in males and females aged 18 to 70.

Topical application of the 300 mM dose resulted in a significant (p=0.0045) improvement in the appearance of M6P treated skin compared to placebo treated skin over the 28 day study period post-damage, as quantitatively measured by comparison of median ΔE (see FIG. 1). Comparison of quantitative measures of skin redness (Δa) within subjects (i.e. M6P versus placebo treated graft sites within the same individual) demonstrated that over the first week post-grafting M6P treatment reduced skin redness in 74.4% of subjects (see FIG. 2). M6P treatment also resulted in a significant (p<0.05) reduction in skin darkness, i.e. an improvement in skin lightness such that it blends better with surrounding normal skin (ΔL) versus placebo treatment over the first two weeks post-damage (see FIG. 3).

Data from subjects dosed topically with 600 mM M6P also demonstrated a significant (p=0.0003) improvement in skin appearance over the 28 day study period (see FIG. 4), a consistently significant (p<0.05) reduction in skin darkness, i.e. an improvement in skin lightness such that it blends better with surrounding normal skin (ΔL) over the first two weeks post-damage (see FIG. 5) and a reduction in redness (Δa) in 84.6% of subjects in the first week post-damage (see FIG. 6). These reductions in redness with 600 mM topical M6P treatment were even further marked than with the 300 mM topical M6P treatment.

Topical applications of M6P at 300 mM and 600 mM had a favourable safety profile in this clinical study comparable to that of placebo treatment in terms of systemic serious adverse or adverse events, local adverse events, local toleration (sensation, erythema, oedema and exudate), laboratory parameters (clinical chemistry, haematology and urinalysis) or vital signs.

The results provided show that mannose-6-phosphate improved a number of endpoints with relevance to a variety of dermatological indications, including: improvement of skin appearance, a reduction of redness and an improvement in skin lightness such that damaged skin treated with mannose-6-phosphate blends better with the surrounding normal skin than placebo treated skin. Damaged skin treated with mannose-6-phosphate has an improved appearance and blends better with the surrounding normal skin.

The data shows the cosmetic and therapeutic utility of mannose-6-phosphate in situations in improving the appearance and/or reducing redness of the skin (for example following skin resurfacing procedures, such as medium or deep skin peels).

1. Mannose-6-phosphate, or a pharmaceutically or cosmetically acceptable salt, precursor, or analogue thereof, for use in reducing redness of skin.

2. Mannose-6-phosphate, or a pharmaceutically or cosmetically acceptable salt, precursor, or analogue thereof according to claim 1, wherein the skin has been subject to damage, particularly damage to the epidermis and optionally the dermis.

3. Mannose-6-phosphate, or a pharmaceutically or cosmetically acceptable salt, precursor, or analogue thereof according to claim 2, wherein the damage results from a skin resurfacing procedure.

4. Mannose-6-phosphate, or a pharmaceutically or cosmetically acceptable salt, precursor, or analogue thereof according to claim 1, wherein the mannose-6-phosphate, or a cosmetically acceptable salt, precursor, or analogue thereof, is formulated as a cosmetic composition.

5. Mannose-6-phosphate, or a pharmaceutically or cosmetically acceptable salt, precursor, or analogue thereof according to claim 4, wherein the cosmetic composition is for application to the skin by topical administration.

6. Mannose-6-phosphate, or a pharmaceutically or cosmetically acceptable salt, precursor, or analogue thereof, for use in providing and/or maintaining a consistent skin colour.

7. Mannose-6-phosphate, or a pharmaceutically or cosmetically acceptable salt, precursor, or analogue thereof, for use as a skin improvement agent for providing a cosmetic effect.

8. Mannose-6-phosphate, or a pharmaceutically or cosmetically acceptable salt, precursor, or analogue thereof according to claim 7, wherein the skin improvement agent improves macroscopic properties of the skin.

9. Mannose-6-phosphate, or a pharmaceutically or cosmetically acceptable salt, precursor, or analogue thereof according to claim 7, wherein the skin improvement agent improves the visual appearance and/or feel (especially visual appearance) of the skin.

10. Mannose-6-phosphate, or a pharmaceutically or cosmetically acceptable salt, precursor, or analogue thereof according to claim 7, wherein the skin improvement agent improves skin that has been subject to damage, particularly damage to the epidermis and optionally the dermis.

11. Mannose-6-phosphate, or a pharmaceutically or cosmetically acceptable salt, precursor, or analogue thereof according to claim 10, wherein the damage results from a skin resurfacing procedure.

12. Mannose-6-phosphate, or a pharmaceutically or cosmetically acceptable salt, precursor, or analogue thereof according to claim 7, wherein the skin improvement agent provides and/or maintains a consistent skin colour.

13. Mannose-6-phosphate, or a pharmaceutically or cosmetically acceptable salt, precursor, or analogue thereof according to claim 7, wherein the skin improvement agent reduces redness of the skin.

14. Mannose-6-phosphate, or a pharmaceutically or cosmetically acceptable salt, precursor, or analogue thereof according to claim 7, wherein the mannose-6-phosphate, salt, precursor; or analogue thereof, is formulated as a cosmetic composition.

15. Mannose-6-phosphate, or a pharmaceutically or cosmetically acceptable salt, precursor, or analogue thereof according to claim 14, wherein the cosmetic composition is for application to the skin by topical administration.

16. Use of mannose-6-phosphate, or a salt, precursor, or analogue thereof, for reducing redness of skin.

17. Use of mannose-6-phosphate, or a salt, precursor, or analogue thereof, for providing and/or maintaining a consistent skin colour.

18. Use of mannose-6-phosphate, or a cosmetically acceptable salt, precursor, or analogue thereof, as a skin improvement agent for providing a cosmetic effect.

19. A cosmetic method for improving the skin of a subject, wherein the method comprises the step of administering an effective amount of mannose-6-phosphate, or a cosmetically acceptable salt, precursor, or analogue thereof, to the skin of the subject so as to provide a cosmetic effect to the skin.

20. A cosmetic composition comprising mannose-6-phosphate, or a cosmetically acceptable salt, precursor, or analogue thereof and a cosmetically acceptable carrier, wherein the carrier comprises around 1% to 50% by weight of the mannose-6-phosphate, or cosmetically acceptable salt, precursor, or analogue thereof.

21. A cosmetic composition comprising mannose-6-phosphate, or a cosmetically acceptable salt, precursor, or analogue thereof, and a cosmetically acceptable carrier, wherein the carrier comprises around 1% to 5% by weight of the mannose-6-phosphate, or cosmetically acceptable salt, precursor, or analogue thereof.

22. A cosmetic composition comprising mannose-6-phosphate, or a cosmetically acceptable salt, precursor, or analogue thereof, and a cosmetically acceptable carrier, wherein the carrier comprises around 1% to 3% by weight of the mannose-6-phosphate, or cosmetically acceptable salt, precursor, or analogue thereof.

23. A cosmetic composition comprising mannose-6-phosphate, or a cosmetically acceptable salt, precursor, or analogue thereof, and a cosmetically acceptable carrier, wherein the carrier comprises greater than 9% by weight, for example a range of greater than 9% to 50% by weight, of the mannose-6-phosphate, or cosmetically acceptable salt, precursor, or analogue thereof.

24. A chemical skin peel composition comprising mannose-6-phosphate, or a cosmetically acceptable salt, precursor, or analogue thereof, and an active chemical skin peel ingredient.

25. A chemical facial skin peel composition comprising mannose-6-phosphate, or a cosmetically acceptable salt, precursor, or analogue thereof, and an active chemical facial skin peel ingredient.

26. Mannose-6-phosphate, or a pharmaceutically acceptable salt, precursor, or analogue thereof, for use in treating skin that has been subject to damage, particularly damage to the epidermis and optionally the dermis.

27. Mannose-6-phosphate, or a pharmaceutically or cosmetically acceptable salt, precursor, or analogue thereof, for use according to claim 26, wherein the damage results from a skin resurfacing procedure.

28. Mannose-6-phosphate, or a pharmaceutically or cosmetically acceptable salt, precursor, or analogue thereof, for use according to claim 26, wherein for providing and/or maintaining a consistent skin colour.

29. Mannose-6-phosphate, or a pharmaceutically or cosmetically acceptable salt, precursor, or analogue thereof, for use according to claim 26, for reducing redness of skin.

30. Mannose-6-phosphate, or a pharmaceutically acceptable salt, precursor, or analogue thereof, for inhibiting the growth of bacteria on damaged skin.

31. Mannose-6-phosphate, or a pharmaceutically acceptable salt, precursor, or analogue thereof, for reducing swelling of damaged skin.
